(19) United States
(12) Patent Application Publication
(10) Pub. No.: US 2020/0255357 A1
(43) Pub. Date: Aug. 13, 2020

(54) INTEGRATED PROCESS FOR MAKING ALPHA, BETA-UNSATURATED FUNCTIONAL COMPOUND

(71) Applicant: ExxonMobil Research and Engineering Company, Annandale, NJ (US)

(72) Inventor: Kun Wang, Bridgewater, NJ (US)

(21) Appl. No.: 16/743,181

(22) Filed: Jan. 15, 2020

Related U.S. Application Data
(60) Provisional application No. 62/802,748, filed on Feb. 8, 2019.

(51) Int. Cl. C07C 2/86; C07C 51/377; C07C 253/30; C07C 407/00; C07C 1/24

(52) U.S. Cl. CPC C07C 2/862; C07C 51/377; C07C 409/04; C07C 407/00; C07C 1/24; C07C 253/30

(57) ABSTRACT

Provided are processes for preparing alpha, beta-unsaturated functional compounds using four major reaction steps: 1) air oxidation of an iso-paraffin to a mixture of alkyl hydroperoxide and alcohol; 2) converting the alkyl hydroperoxide and alcohol to dialkyl peroxide; 3) oxidative cross-coupling between a primary or secondary alcohol and a compound comprising at least one R3CH2- (R3=hydrogen or an optionally substituted hydrocarbyl) moiety to afford a coupled product using the dialkyl peroxide as a radical initiator, while the dialkyl peroxide is converted to a tertiary alcohol; 4) dehydration of the coupled product to yield an alpha, beta-unsaturated functional compound.

### CROSS-REFERENCE TO RELATED APPLICATION

This application claims the benefit of U.S. Provisional Application No. 62/802748, filed on Feb. 8, 2019, the entire contents of which are incorporated herein by reference.

### FIELD

The present disclosure relates to an integrated process for making alpha, beta-unsaturated functional compounds with concurrent light paraffin upgrading. The process comprises four major reaction steps: 1) air oxidation of an iso-paraffin to a mixture of alkyl hydroperoxide and alcohol; 2) converting the alkyl hydroperoxide and alcohol to dialkyl peroxide; 3) oxidative cross-coupling between a primary or secondary alcohol and a compound comprising at least one R₃CH₂- moiety, wherein R₃ is hydrogen or hydrocarbyl, to produce a coupled product using the dialkyl peroxide as a radical initiator, while the dialkyl peroxide is converted to a tertiary alcohol; 4) dehydration of the coupled product to yield an alpha, beta-unsaturated functional compound.

### BACKGROUND

Due to their high reactivity and versatile applicability, alpha, beta-unsaturated functional compounds have become key products in industrial chemistry. One important subset of alpha, beta-unsaturated functional compounds is vinylic compounds. The extensive group of vinylic compounds includes products such as styrene, acrylic acid, vinyl chloride, vinyl acetate and acrylonitrile.

For example, acrylic acid and acrylonitrile are important chemicals/intermediates for a variety of applications. Acrylic acid is widely used in plastics, coatings, adhesives, elastomers, as well as floor polishes, and paints. Acrylonitrile is used principally as a monomer to prepare polyacrylonitrile, a homopolymer, or several important copolymers, such as styrene-acrylonitrile (SAN), acrylonitrile butadiene styrene (ABS), acrylonitrile styrene acrylate (ASA), and other synthetic rubbers such as acrylonitrile butadiene (NBR).

Dimerization of acrylonitrile affords adiponitrile, used in the synthesis of certain polyamides. Small amounts are also used as a fumigant. Acrylonitrile and derivatives, such as 2-chloro-acrylonitrile, are dienophiles in Diels-Alder reactions. Acrylonitrile is also a precursor in the industrial manufacture of acrylamide and acrylic acid. Both acrylic acid and acrylonitrile are projected to have a robust growth rate as global demand continues to rise.

Currently vinylic compounds such as acrylic acid and acrylonitrile are formed by oxidation (acrylic acid) or ammoxidation (acrylonitrile) of propylene using solid oxide catalysts. Both processes require high purity propylene as feed and use complex mixtures of oxide as catalyst for the gas phase oxidation or ammoxidation.

Acrylic acid is currently produced via propylene oxidation in two steps. In a first step propylene is oxidized to acrolein and in a second step the acrolein is further oxidized to acrylic acid.

Accordingly, there is a need to identify new methods to produce alpha, beta-unsaturated functional compounds, particularly methods that utilize readily available and low cost feedstocks and which may avoid the use of complex catalysts.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

### SUMMARY

Disclosed herein is a novel, integrated process for making alpha, beta-unsaturated functional compounds such as styrene, vinyl chloride, acrylic acid, acrylonitrile, and the like, with concurrent light paraffin upgrading. The process comprises four major reaction steps: 1) air oxidation of an iso-paraffin to a mixture of alkyl hydroperoxide and alcohol; 2) converting the alkyl hydroperoxide and alcohol to dialkyl peroxide; 3) oxidative cross-coupling between a primary or secondary alcohol and a compound comprising least one R₃CH₂- moiety, wherein R₃ is hydrogen or optionally substituted hydrocarbyl, to produce a coupled product, using the dialkyl peroxide as a radical initiator, while the dialkyl peroxide is converted to a tertiary alcohol; 4) dehydration of the coupled product to yield an alpha, beta-unsaturated functional compound. The net reaction is oxidatively converting the compound comprising at least one R₃CH₂- moiety, wherein R₃ is hydrogen or optionally substituted hydrocarbyl, to an alpha, beta-unsaturated functional compound using iso-paraffin and air, while the iso-paraffin is upgraded to a tertiary alcohol. The tertiary alcohol may be recovered as a chemical, or dehydrated to iso-olefin, or used as high-octane gasoline blend.

In one aspect the present disclosure provides a process for making an alpha, beta-unsaturated functional compound, said process comprising:
(a) oxidizing a first feed stream comprising one or more iso-paraffins to form alkyl hydroperoxides and first tertiary alcohols;
(b) catalytically converting the alkyl hydroperoxides and first tertiary alcohols to dialkyl peroxides;
(c) oxidatively coupling a primary or secondary alcohol and a compound comprising at least one R₃CH₂- moiety, wherein R₃ is hydrogen or optionally substituted hydrocarbyl, using the dialkyl peroxides as a radical initiator to afford a coupled product comprising at least one hydroxyl group, while the dialkyl peroxides are converted to second tertiary alcohols; and
(d) dehydrating the coupled product to afford an alpha, beta-unsaturated functional compound.

In some embodiments the first feed stream comprises an iso-paraffin selected from the group consisting of iso-butane, iso-pentane, iso-hexane, iso-heptane and mixtures thereof.

In some preferred embodiments the first feed stream comprises iso-butane and the first tertiary alcohol is t-butyl alcohol.

In some embodiments the primary alcohol has formula R₁CH₂OH, wherein R₁ is H or optionally substituted hydrocarbyl.

In some embodiments the secondary alcohol has formula R₁R₂CHOH, wherein R₁ and R₂ are independently selected from optionally substituted hydrocarbyl.

Preferred primary alcohols may be selected from group consisting of methanol, ethanol, n-propanol and n-butanol.

Preferred secondary alcohol may be selected from 2-propanol and sec-butanol.

In some embodiments the compound comprising at least one R₃CH₂- moiety may be represented by the formula CH₃X, wherein X is selected from the group consisting of optionally substituted hydrocarbyl, carbonyl, carboxylate, amino, halo, cyano, hydroxyl, thiol, nitro, sulfonate, phosphonate and borato.

Exemplary optionally substituted hydrocarbyl groups comprise optionally substituted aliphatic and optionally substituted aromatic groups.

In one preferred embodiment the compound represented by the formula CH₃X comprises methyl substituted aromatic compounds of formula Ar(CH₃)ₓ where x=1, 2 or 3 and Ar represents an optionally substituted aryl ring system. Particularly preferred methyl substituted aromatic compounds may be selected from the group consisting of toluene, xylene, mesitylene and mixtures thereof.

When the compound of formula CH₃X comprises methyl substituted aromatic compounds and the primary alcohol is methanol, the presently disclosed integrated process yields vinyl substituted aromatic compounds, of general formula ArCH═CH₂.

For example, when CH₃X is toluene and the primary alcohol is methanol, the product of the present integrated process is styrene. When CH₃X is xylene and the primary alcohol is methanol the product is vinyl toluene and/or divinyl benzene.

In another preferred embodiment the compound of formula CH₃X comprises methyl substituted cycloalkanes. Particularly preferred methyl substituted cycloalkanes may be selected from the group consisting of methyl cyclobutane, methyl cyclopentane, methyl cyclohexane, methyl cycloheptane and methyl cyclooctane.

When the compound of formula CH₃X comprises methyl substituted cycloalkanes and the primary alcohol is methanol, the presently disclosed integrated process yields vinyl substituted cycloalkanes.

For example, when CH₃X is methyl cyclohexane and the primary alcohol is methanol, the product of the present integrated process is vinyl cyclohexane.

In another preferred embodiment the compound of formula CH₃X comprises carboxylic acids. Particularly preferred carboxylic acids may be selected from the group consisting of acetic acid, propanoic acid and butanoic acid.

When the compound of formula CH₃X is acetic acid and the primary alcohol is methanol, the presently disclosed integrated process yields acrylic acid.

In another preferred embodiment the compound of formula CH₃X comprises alkyl halides. Particularly preferred alkyl halides include alkyl chlorides such as chloromethane.

When the compound of formula CH₃X is chloromethane and the primary alcohol is methanol, the presently disclosed integrated process yields vinyl chloride.

In another preferred embodiment the compound CH₃X comprises alkyl cyanides. When the compound of formula CH₃X is acetonitrile and the primary alcohol is methanol, the presently disclosed integrated process yields acrylonitrile.

In another preferred embodiment the compound of formula CH₃X comprises alcohols. Particularly preferred alcohols include ethanol, n-propanol and n-butanol.

When the compound of formula CH₃X is methanol and the primary alcohol is also methanol, the presently disclosed integrated process yields vinyl alcohol.

In other embodiments the compound comprising at least one R₃CH₂- moiety may be represented by the formula R₃CH₂X, wherein R₃ is optionally substituted hydrocarbyl, and wherein X is selected from the group consisting of optionally substituted hydrocarbyl, carbonyl, carboxylate, amino, halo, cyano, hydroxyl, thiol, nitro, sulfonate, phosphonate and borato.

The presently disclosed process may offer one or more of the following advantages:
utilizes the abundant supply of light paraffins, particularly C2-C5 paraffins, in North America
a wide range of useful alpha, beta-unsaturated functional compounds may be prepared
the process operates under relatively mild conditions
expensive and often toxic transition metal based catalysts may be avoided.
light paraffins may be upgraded to higher value alcohols

Further features and advantages of the present disclosure will be understood by reference to the following drawings and detailed description.

### BRIEF DESCRIPTION OF THE DRAWINGS

FIG. 1 is a flow scheme of a process according to one embodiment of the present disclosure.

### DETAILED DESCRIPTION OF THE EMBODIMENTS

The following is a detailed description of the disclosure provided to aid those skilled in the art in practicing the present disclosure. Those of ordinary skill in the art may make modifications and variations in the embodiments described herein without departing from the spirit or scope of the present disclosure.

Although any methods and materials similar or equivalent to those described herein can also be used in the practice or testing of the present disclosure, the preferred methods and materials are now described.

It must also be noted that, as used in the specification and the appended claims, the singular forms ‘a’, ‘an’ and ‘the’ include plural referents unless otherwise specified. Thus, for example, reference to ‘vinylic compound’ may include more than one vinylic compound, and the like.

Throughout this specification, use of the terms ‘comprises’ or ‘comprising’ or grammatical variations thereon shall be taken to specify the presence of stated features, integers, steps or components but does not preclude the presence or addition of one or more other features, integers, steps, components or groups thereof not specifically mentioned.

As used herein the term ‘alpha, beta-unsaturated functional compound’ includes compounds of general formula X—C(Rₐ)═C(R_{b})(R_{c}) wherein Rₐ, R_{b} and R_{c} are independently selected from hydrogen or optionally substituted hydrocarbyl and X is selected from, for example, optionally substituted hydrocarbyl, carbonyl, carboxylate, amino, halo, cyano, hydroxyl, thiol, nitro, sulfonate, phosphonate and borato

‘Optional’ or ‘optionally’ means that the subsequently described event or circumstance may or may not occur, and that the description includes instances where said event or circumstance occurs and instances where it does not. For example, the phrase “optionally substituted hydrocarbyl” means that a hydrocarbyl moiety may or may not be substituted and that the description includes both unsubstituted hydrocarbyl and hydrocarbyl where there is substitution.

As used herein the term ‘hydrocarbyl’ refers to hydrocarbyl radicals containing 1 to about 50 carbon atoms, specifically 1 to about 24 carbon atoms, most specifically 1 to about 16 carbon atoms, including branched or unbranched, saturated or unsaturated species, such as alkyl groups, alkenyl groups, aryl groups, and the like. ‘Substituted hydrocarbyl’ refers to hydrocarbyl substituted with one or more substituent groups.

The term ‘substituted hydrocarbyl’ includes ‘substituted alkyl’, ‘substituted alkenyl’, ‘substituted aryl’ and the like. Substitution means at least one hydrogen atom bound to a carbon atom is replaced with substituents such as hydroxyl, alkoxy, alkylthio, phosphino, amino, halo, silyl, cyano, borato, carbonyl, carboxyl and the like.

Unless specifically stated or obvious from context, as used herein, the term “about” is understood as within a range of normal tolerance in the art, for example within two standard deviations of the mean. ‘About’ can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein in the specification and the claim can be modified by the term ‘about’.

Any methods provided herein can be combined with one or more of any of the other methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The presently disclosed integrated process comprises four major reaction steps: 1) air oxidation of an iso-paraffin to a mixture of alkyl hydroperoxide and alcohol; 2) converting the alkyl hydroperoxide and alcohol to dialkyl peroxide; 3) oxidative cross-coupling between a primary or secondary alcohol and a compound comprising at least one R₃CH₂- moiety, wherein R₃ is hydrogen or optionally substituted hydrocarbyl, to produce a coupled product, using the dialkyl peroxide as a radical initiator, while the dialkyl peroxide is converted to a tertiary alcohol; 4) dehydration of the coupled product to yield an alpha, beta-unsaturated functional compound.

The chemistry of Steps 1 and 2 with respect to iso-butane feed is shown below in corresponding reactions 1 and 2:

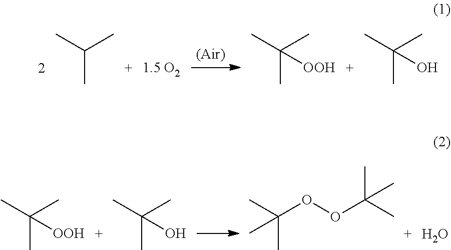

Reaction 1 (Step 1) represents the air oxidation of iso-butane to t-butylhydroperoxide and t-butyl alcohol.

Reaction 2 (Step 2) illustrates conversion of t-butylhydroperoxide and t-butyl alcohol to di-t-butyl peroxide (DTBP) using an acid catalyst.

### Reaction Steps 3 and 4

Reaction 3A represents one embodiment of Step 3, being the oxidative cross-coupling between a primary alcohol and a compound of formula CH₃X.

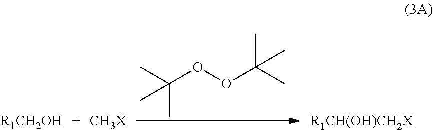

Reaction 4A represents one embodiment of Step 4, being the dehydration of the coupled product of reaction 3A.

R₁CH(OH)CH₂X→R₁CH═CHX+H₂O (4A)

Reaction 3B represents another embodiment of Step 3, being the oxidative cross-coupling between a secondary alcohol and a compound of formula CH₃X.

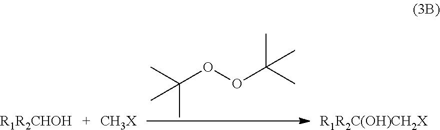

Reaction 4B represents another embodiment of Step 4, being the dehydration of the coupled product of reaction 3B.

R₁R₂C(OH)CH₂X→R₁R₂C═CHX+H₂O (4B)

Reaction 3C represents another embodiment of Step 3, being the oxidative cross-coupling between a primary alcohol and a compound of formula R₃CH₂X.

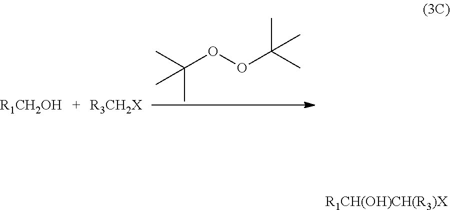

Reaction 4C represents another embodiment of Step 4, being the dehydration of the coupled product of reaction 3C.

R₁CH(OH)CH(R₃)X→R₁CH═C(R₃)X+H₂O (4C)

Reaction 3D represents another embodiment of Step 3, being the oxidative cross-coupling between a secondary alcohol and a compound of formula R₃CH₂X.

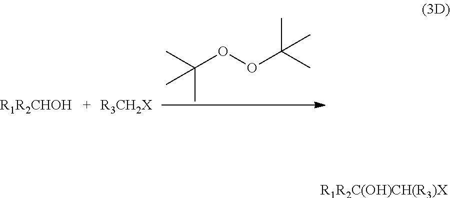

Reaction 4D represents another embodiment of Step 4, being the dehydration of the coupled product of reaction 3D.

R₁R₂C(OH)CH(R₃)X→R₁R₂C═C(R₃)X+H₂O (4D)

Reaction 3E below, illustrates the oxidative cross-coupling between methanol and a compound CH₃X to form a primary alcohol.

CH₃X+CH₃OH→X—CH₂—CH₂—OH (3E)

Reaction 4E below illustrates dehydration of the primary alcohol to yield a vinylic product.

X—CH₂—CH₂—OH→X—CH═CH₂+H₂O (4E)

The following describes several exemplary embodiments of Reactions (3) and (4).

In one exemplary embodiment Reaction (3F) illustrates the oxidative cross-coupling between methanol and acetic acid using DTBP to afford 3-hydroxy propanoic acid.

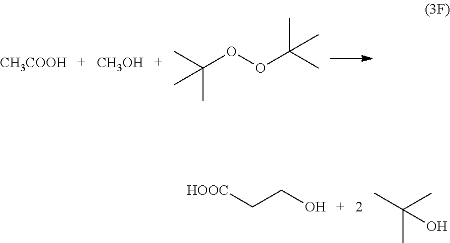

Reaction (4F) illustrates dehydration of the 3-hydroxy propanoic acid to afford acrylic acid.

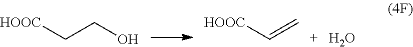

In another exemplary embodiment Reaction (3G) illustrates the oxidative cross-coupling between methanol and acetonitrile using DTBP to afford 2-cyanoethanol.

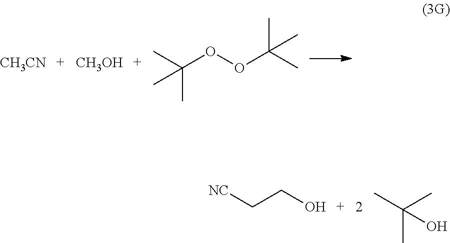

Reaction (4G) illustrates dehydration of the 2-cyanoethanol to afford acrylonitrile.

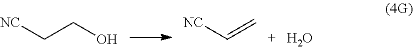

In another exemplary embodiment Reaction (3H) illustrates the oxidative cross-coupling between methanol and toluene using DTBP to afford (2-hydroxy ethyl) benzene.

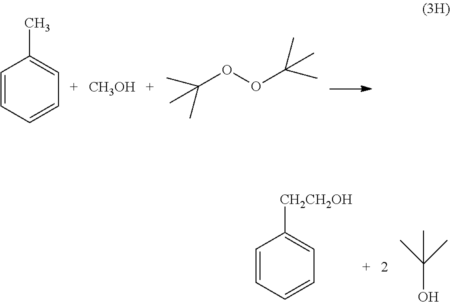

Reaction (4H) illustrates dehydration of the (2-hydroxy ethyl) benzene to afford styrene.

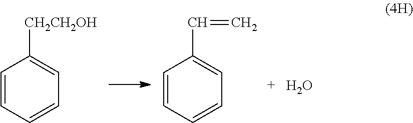

In another exemplary embodiment Reaction (3I) illustrates the oxidative cross-coupling between methanol and chloromethane using DTBP to afford 2-chloroethanol.

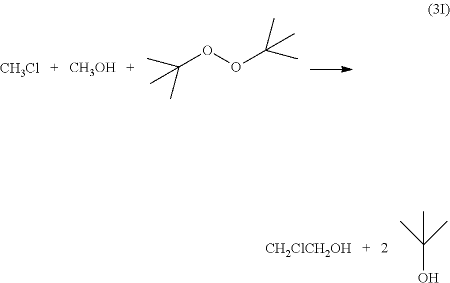

Reaction (4I) illustrates dehydration of the 2-chloroethanol to afford vinyl chloride.

CH₂ClCH₂OH→CHCl═CH₂+H₂O (4I)

Steps 1 and 2 have been previously described in applicant's co-pending application, U.S. App. Publ. No. 2017/0101366, incorporated by reference herein in its entirety. U.S. App. Publ. No. 2017/0101366 describes a process to couple functional molecules into di-functional or multi-functional molecules using dialkyl peroxide as a radical initiator. Whereas U.S. App. Publ. No. 2017/0101366 is directed to create di-functional or multi-functional molecules utilizing coupling reactions, the present disclosure utilizes dialkyl peroxide to initiate oxidative coupling of a primary or secondary alcohol with a compound comprising at least one R₃CH₂- moiety to afford a coupled product comprising at least one hydroxyl group which is subsequently dehydrated to an alpha, beta-unsaturated functional compound.

Iso-butane oxidation in Step 1/Reaction 1 is commercially well-established for making t-butyl hydroperoxide (TBHP) for propylene oxide manufacture. Variants of the process are described, for example, in U.S. Pat. Nos. 2,845,461; 3,478,108; 4,408,081 and 5,149,885. EP 0567336 and U.S. Pat. No. 5,162,593 disclose co-production of TBHP and t-butyl alcohol (TBA).

As TBA is one of the reactants used in Step 2 of the present disclosure, the present integrated process scheme utilizes Step 1 as a practical source of these two reactants. Air (approximately 21% oxygen), a mixture of nitrogen and oxygen containing 2-20 vol % oxygen, or pure oxygen, can be used for the oxidation, as long as the oxygen-to-hydrocarbon vapor ratio is kept outside the explosive regime. Preferably air is used as the source of oxygen.

Step 1/Reaction 1 is preferably carried out at a temperature from about 110 to about 150° C., more preferably from about 130 to about 140° C.

The pressure is preferably from about 300 to about 800 psig, more preferably from about 450 to about 550 psig.

The reaction time may be from about 2 hours to about 24 hours, preferably from about 6 hours to about 8 hours. Such reaction times typically produce conversions from about 15% to about 70%, preferably from about 30 to about 50%.

Typically, selectivity to TBHP is from about 50 to about 80%, and to TBA from about 20 to about 50%.

In Step 2/Reaction 2, the conversion of the TBHP and TBA to di-t-butyl peroxide (DTBP) is performed using an acid catalyst. For example, U.S. Pat. No. 5,288,919 describes the use of an inorganic heteropoly and/or isopoly acid catalyst (such as for the reaction of TBA with TBHP). The concurrent production of DTBP and TBA from TBHP is also described in U.S. Pat. No. 5,345,009.

A preferred configuration for Step 2 of the presently disclosed integrated process uses reactive distillation in which product water is continuously removed as an overhead by-product.

Step 2 is preferably carried out at a temperature from about 50 to about 200° C., more preferably from about 60 to about 150° C., even more preferably from about 80 to about 120° C.

Pressure for the reaction is held within appropriate ranges to ensure the reaction occurs substantially in the liquid phase, for example, from about 0 to about 300 psig, preferably from about 5 to about 100 psig, more preferably from about 15 to about 50 psig.

The TBHP to TBA mole ratio may be in the range from about 0.5 to about 2, preferably from about 0.8 to about 1.5, more preferably from about 0.9 to about 1.1. The reaction may be performed with or without a solvent. Suitable solvents comprise hydrocarbons having a carbon number greater than 3. Suitable solvents include paraffins, cycloalkanes, or aromatics.

Advantageously, the unreacted iso-butane from Step 1 may be used as a solvent for Step 2.

An acid catalyst such as Amberlyst™ resin, Nafion™ resin, aluminosilicates, acidic clay, zeolites (natural or synthetic), silicoaluminophosphates (SAPO), heteropolyacids, acidic oxides such as tungsten oxide on zirconia, molybdenum oxide on zirconia, sulfonated zirconia, liquid acids such sulfuric acid, or acidic ionic liquids may be used in Step 2/Equation 2 to promote the conversion of TBHP and TBA into DTBP.

Reaction (3) is preferably carried out at a temperature from about 100 to about 170° C., more preferably from about 130 to about 150° C. The pressure is preferably from about 100 to about 1500 psig, more preferably from about 500 to about 1200 psig. The mole ratio of primary or secondary alcohol to the compound comprising at least one R₃CH₂- moiety may be from about 0.1 to about 20, preferably from about 0.5 to about 10, more preferably from about 1 to about 5. The mole ratio of primary or secondary alcohol plus the compound comprising at least one R₃CH₂- moiety to DTBP may be from about 100 to about 1, preferably from about 50 to about 2.

The reaction time may be from about 2 hours to about 24 hours, preferably from about 4 hours to about 16 hours.

Complete conversion of DTBP is typically achieved in Reaction (3).

The group X in the compounds CH₃X or R₃CH₂X may be selected from optionally substituted hydrocarbyl, halogen, —OH (hydroxyl), —CN (cyano), —C(O)OH (carboxylic), —NHR′ (amino, where R′ may be H or a hydrocarbyl group), —SH (mercapto), —NO₂ (nitro), —OSO₃H (sulfonato), —OPO₃H (phosphato), —OBO (borato), and the like.

In Reaction (4) the coupled product, for example, HOCH₂CH₂X, is dehydrated to give the desired vinylic compound CH₂═CHX. For example, a solid acid catalyst is used in the reaction zone to dehydrate HOCH₂CH₂X to give CH₂═CHX.

Representative solid acid catalysts comprise acidic clays, amorphous aluminosilicates, acidic alumina such as gamma-alumina or chloride-alumina and fluoride alumina, zeolites, silicoaluminophosphates, acidic mixed metal oxides such as tungsten oxide/zirconium oxide, molybdenum oxide/zirconium oxide, acidic resins such as Nafion™, Dowex™, Amberlyst™, supported phosphoric acid, etc. The acid can also be a Lewis acid, such as BF₃, BCl₃, AlCl₃, either neat or supported on a solid support.

Reaction (4) is preferably carried out at a temperature from about 50 to about 450° C., more preferably from about 100 to about 350° C., most preferably from about 100 to about 250° C.

Pressure is preferably from about 0 psig to about 2000 psig, more preferably from about 50 to about 1500 psig, most preferably from about 100 to about 1000 psig.

In one example, the overall reaction stoichiometry from equations 1 to 4 is shown below in Equation 5.

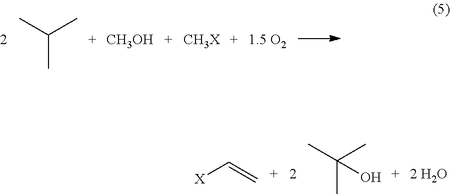

The net effect is coupling of methanol with CH₃X or R₃CH₂X (X is a functional group) to yield an alpha, beta-unsaturated functional compound using iso-butane as an oxygen carrier, while iso-butane is converted to t-butyl alcohol—an upgraded product from isobutane.

In the alpha, beta-unsaturated functional compound, the unsaturation (C═C bond) comes from the primary or secondary alcohol after dehydration of the coupled product and the functional group X comes from CH₃X or R₃CH₂X. In some embodiments, the functionality X can be a carboxylate group —C(O)OH which yields acrylic acid, cyano group —CN which yields acrylonitrile, halide, such as chloride —Cl, which yields vinyl chloride, hydroxyl group —OH which yields vinyl alcohol, amino —NHR′ which yields vinyl amine, (where R′ may be H or a hydrocarbyl group), phenyl group which yields styrene, —SH which yields vinyl mercaptan, —NO₂ which yields nitroethylene, —OSO₃H which yields vinyl sulfonic acid, —OPO₃H₂ which yields vinyl phosphoric acid, —OBO which yields vinyl borate, and so forth.

Depending on the nature of the iso-paraffin, the resulting alcohol can be used as high octane blend for gasoline: e.g., t-butyl alcohol from iso-butane, or 2-methyl-2-butanol from iso-pentane. Alternatively, the alcohols can be converted to olefins as chemical products via dehydration (e.g., iso-butylene), or etherified with an alcohol such as methanol or ethanol making ether as gasoline blend (e.g., MTBE or ETBE from isobutane).

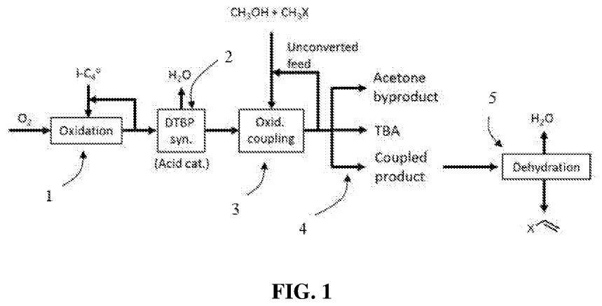

FIG. 1 illustrates a process scheme according to one embodiment of the present disclosure in which iso-butane is the feed for the initial oxidation step. A feed comprising iso-butane) (i-C₄°) is sent to an oxidation reactor (1) to which an oxidizing gas comprising O₂ is also fed.

The oxidation mixture comprising t-butyl hydroperoxide (TBHP) and t-butyl alcohol (TBA) is sent to the next reactor (2) after i-C4° is separated and optionally recycled to the oxidation reactor (1), where di-t-butyl peroxide (DTBP) is formed over an acid catalyst (for example Amberlyst™ or acidic clay). A preferred configuration for this reactor is reactive distillation in which the co-product water is continuously removed (as illustrated).

DTBP is sent to the next reactor (3) to initiate oxidative coupling of CH₃OH and CH₃X.

The reaction products are separated/fractionated in the next step (4). Unconverted feed is recycled to the oxidative coupling reactor (3). Final products from this process include primary alcohol as a result of the oxidative coupling of CH₃OH and CH₃X, t-butyl alcohol (TBA) and by-product acetone.

The coupled product is then sent to reactor (5) where it is dehydrated to afford the vinylic product.

### Certain Embodiments

Certain embodiments of processes according to the present disclosure are presented in the following paragraphs.

Embodiment 1 provides a process for making alpha, beta-unsaturated functional compounds, said process comprising:
(a) oxidizing a first feed stream comprising one or more iso-paraffins to form alkyl hydroperoxides and first tertiary alcohols;
(b) catalytically converting the alkyl hydroperoxides and first tertiary alcohols to dialkyl peroxides;
(c) oxidatively coupling a primary or secondary alcohol and a compound comprising at least one R₃CH₂- moiety, wherein R₃ is hydrogen or optionally substituted hydrocarbyl, using the dialkyl peroxides as a radical initiator to afford a coupled product comprising at least one hydroxyl group, while the dialkyl peroxides are converted to second tertiary alcohols; and
(d) dehydrating the coupled product to afford an alpha, beta-unsaturated functional compound.

Embodiment 2 provides a process according to Embodiment 1, wherein the first feed stream comprises an iso-paraffin selected from the group consisting of iso-butane, iso-pentane, iso-hexane, iso-heptane and mixtures thereof.

Embodiment 3 provides a process according to Embodiment 1 or Embodiment 2, wherein the first feed stream comprises iso-butane.

Embodiment 4 provides a process according to any one of Embodiments 1 to 3, wherein the primary alcohol has formula R₁CH₂OH, wherein R₁ is H or optionally substituted hydrocarbyl.

Embodiment 5 provides a process according to any one of Embodiments 1 to 3, wherein the secondary alcohol has formula R₁R₂CHOH, wherein R₁ and R₂ are independently selected from optionally substituted hydrocarbyl.

Embodiment 6 provides a process according to any one of Embodiments 1 to 4, wherein the primary alcohol is selected from group consisting of methanol, ethanol, n-propanol or n-butanol.

Embodiment 7 provides a process according to any one of Embodiments 1 to 3 or 5, wherein the secondary alcohol is selected from 2-propanol, or sec-butanol.

Embodiment 8 provides a process according to any one of Embodiments 1 to 7, wherein the compound comprising at least one R₃CH₂- moiety is represented by the formula CH₃X, wherein X is selected from the group consisting of optionally substituted hydrocarbyl, carbonyl, carboxylate, amino, halo, cyano, hydroxyl, thiol, nitro, sulfonate, phosphonate and borato.

Embodiment 9 provides a process according to any one of Embodiments 1 to 7, wherein the compound comprising at least one R₃CH₂- moiety is represented by the formula R₃CH₂X, wherein R₃ is optionally substituted hydrocarbyl and wherein X is selected from the group consisting of optionally substituted hydrocarbyl, carbonyl, carboxylate, amino, halo, cyano, hydroxyl, thiol, nitro, sulfonate, phosphonate and borato.

Embodiment 10 provides a process according to Embodiment 8, wherein the optionally substituted hydrocarbyl is an optionally substituted aliphatic group or optionally substituted aromatic group.

Embodiment 11 provides a process according to Embodiment 9, wherein the optionally substituted hydrocarbyl is an optionally substituted aliphatic group or optionally substituted aromatic group.

Embodiment 12 provides a process according to Embodiment 8 or Embodiment 10, wherein the compound CH₃X is a methyl substituted aromatic compound Ar(CH₃)ₓ where x=1, 2 or 3 and Ar is an optionally substituted aromatic ring system.

Embodiment 13 provides a process according to Embodiment 12, wherein the methyl substituted aromatic compound is selected from the group consisting of toluene, xylene and mesitylene.

Embodiment 14 provides a process according to Embodiment 13, wherein the compound CH₃X is toluene.

Embodiment 15 provides a process according to Embodiment 13, wherein the compound CH₃X is xylene.

Embodiment 16 provides a process according to Embodiment 8, wherein the compound CH₃X is a methyl substituted cycloalkane.

Embodiment 17 provides a process according to Embodiment 16, wherein the methyl substituted cycloalkane is selected from the group consisting of methylcyclobutane, methylcyclopentane, methylcyclohexane, methylcycloheptane and methylcyclooctane.

Embodiment 18 provides a process according to Embodiment 17, wherein the compound CH₃X is methylcyclohexane.

Embodiment 19 provides a process according to Embodiment 8, wherein the compound CH₃X is a carboxylic acid.

Embodiment 20 provides a process according to Embodiment 19, wherein the carboxylic acid is selected from acetic acid, propanoic acid and butanoic acid.

Embodiment 21 provides a process according to Embodiment 20, wherein the compound CH₃X is acetic acid.

Embodiment 22 provides a process according to Embodiment 8, wherein the compound CH₃X is an alkyl halide.

Embodiment 23 provides a process according to Embodiment 22, wherein the alkyl halide is selected from alkyl chlorides, preferably chloromethane.

Embodiment 24 provides a process according to Embodiment 23, wherein the compound CH₃X is chloromethane.

Embodiment 25 provides a process according to Embodiment 8, wherein the compound CH₃X is an alkyl cyanide.

Embodiment 26 provides a process according to claim 25, wherein the compound CH₃X is acetonitrile.

Embodiment 27 provides a process according to Embodiment 1 wherein the primary or secondary alcohol is methanol and the alpha, beta-unsaturated functional compound is selected from the group consisting of styrene, vinylcyclohexane, vinyl chloride, vinyl alcohol, acrylonitrile, acrylic acid, vinyl amine, vinyl mercaptan, vinyl sulfonic acid and vinyl borate.

The contents of all references, including published patents and patent applications cited throughout the application are hereby incorporated by reference.

It is understood that the examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

1. A process for making alpha, beta-unsaturated functional compounds, said process comprising:
(a) oxidizing a first feed stream comprising one or more iso-paraffins to form alkyl hydroperoxides and first tertiary alcohols;
(b) catalytically converting the alkyl hydroperoxides and first tertiary alcohols to dialkyl peroxides;
(c) oxidatively coupling a primary or secondary alcohol and a compound comprising at least one R₃CH₂- moiety, wherein R₃ is hydrogen or optionally substituted hydrocarbyl, using the dialkyl peroxides as a radical initiator to afford a coupled product comprising at least one hydroxyl group, while the dialkyl peroxides are converted to second tertiary alcohols; and
(d) dehydrating the coupled product to afford an alpha, beta-unsaturated functional compound.

2. A process according to claim 1, wherein the first feed stream comprises an iso-paraffin selected from the group consisting of iso-butane, iso-pentane, iso-hexane, iso-heptane and mixtures thereof.

3. A process according to claim 1, wherein the first feed stream comprises iso-butane.

4. A process according to claim 1, wherein the primary alcohol has formula R₁CH₂OH, wherein R₁ is H or optionally substituted hydrocarbyl.

5. A process according to claim 1, wherein the secondary alcohol has formula R₁R₂CHOH, wherein R₁ and R₂ are independently selected from optionally substituted hydrocarbyl.

6. A process according to claim 1, wherein the primary alcohol is selected from group consisting of methanol, ethanol, n-propanol or n-butanol.

7. A process according to claim 1, wherein the secondary alcohol is selected from 2-propanol, or sec-butanol.

8. A process according to claim 1, wherein the compound comprising at least one R₃CH₂-moiety is represented by the formula CH₃X, wherein X is selected from the group consisting of optionally substituted hydrocarbyl, carbonyl, carboxylate, amino, halo, cyano, hydroxyl, thiol, nitro, sulfonate, phosphonate and borato.

9. A process according to claim 1, wherein the compound comprising at least one R₃CH₂-moiety is represented by the formula R₃CH₂X, wherein R₃ is optionally substituted hydrocarbyl and wherein X is selected from the group consisting of optionally substituted hydrocarbyl, carbonyl, carboxylate, amino, halo, cyano, hydroxyl, thiol, nitro, sulfonate, phosphonate and borato.

10. A process according to claim 8, wherein the optionally substituted hydrocarbyl is an optionally substituted aliphatic group or optionally substituted aromatic group.

11. A process according to claim 9, wherein the optionally substituted hydrocarbyl is an optionally substituted aliphatic group or optionally substituted aromatic group.

12. A process according to claim 8, wherein the compound CH₃X is a methyl substituted aromatic compound Ar(CH₃)ₓ where x=1, 2 or 3 and Ar is an optionally substituted aromatic ring system.

13. A process according to claim 12, wherein the methyl substituted aromatic compound is selected from the group consisting of toluene, xylene and mesitylene.

14. A process according to claim 13, wherein the compound CH₃X is toluene.

15. A process according to claim 13, wherein the compound CH₃X is xylene.

16. A process according to claim 8, wherein the compound CH₃X is a methyl substituted cycloalkane.

17. A process according to claim 16, wherein the methyl substituted cycloalkane is selected from the group consisting of methylcyclobutane, methylcyclopentane, methylcyclohexane, methylcycloheptane and methylcyclooctane.

18. A process according to claim 17, wherein the compound CH₃X is methylcyclohexane.

19. A process according to claim 8, wherein the compound CH₃X is a carboxylic acid.

20. A process according to claim 19, wherein the carboxylic acid is selected from acetic acid, propanoic acid and butanoic acid.

21. A process according to claim 20, wherein the compound CH₃X is acetic acid.

22. A process according to claim 8, wherein the compound CH₃X is an alkyl halide.

23. A process according to claim 22, wherein the alkyl halide is selected from alkyl chlorides, preferably chloromethane.

24. A process according to claim 23, wherein the compound CH₃X is chloromethane.

25. A process according to claim 8, wherein the compound CH₃X is an alkyl cyanide.

26. A process according to claim 25, wherein the compound CH₃X is acetonitrile.

27. A process according to claim 1, wherein the primary or secondary alcohol is methanol and the alpha, beta-unsaturated functional compound is selected from the group consisting of styrene, vinylcyclohexane, vinyl chloride, vinyl alcohol, acrylonitrile, acrylic acid, vinyl amine, vinyl mercaptan, vinyl sulfonic acid and vinyl borate.
